(19) United States
(12) Patent Application Publication
(10) Pub. No.: US 2017/0251998 A1
(43) Pub. Date: Sep. 7, 2017

(54) ULTRASONIC DIAGNOSTIC DEVICE

(71) Applicant: Hitachi, Ltd., Tokyo (JP)

(72) Inventors: Toshinori MAEDA, Tokyo (JP); Eiji KASAHARA, Tokyo (JP); Noriyoshi MATSUSHITA, Tokyo (JP); Masaru MURASHITA, Tokyo (JP)

(73) Assignee: Hitachi, Ltd., Tokyo (JP)

(21) Appl. No.: 15/510,247

(22) PCT Filed: Aug. 20, 2015
(86) PCT No.: PCT/JP2015/073302

§ 371 Date: Mar. 10, 2017

(30) Foreign Application Priority Data
Sep. 11, 2014 (JP) 2014-185327

(51) Int. Cl. A61B 8/02; A61B 8/08; A61B 8/00

(52) U.S. Cl. CPC A61B 8/02; A61B 8/461; A61B 8/5207; A61B 8/0866; A61B 8/0883

(57) ABSTRACT

A tracking processing unit performs tracking processing for a plurality of states during a tracking period and, on the basis of a plurality of tracking results obtained from the tracking processing for the plurality of states, tracks the movement of a measurement point during the tracking period. In addition, with regard to a plurality of tracking points that comprise the measurement point and an auxiliary point, the tracking processing unit performs tracking processing for each tracking point during the tracking period and, on the basis of a plurality of tracking results obtained from the plurality of tracking points, tracks the movement of the measurement point during the tracking period.

### TECHNICAL FIELD

The present invention relates to an ultrasonic diagnostic device for diagnosing a fetus.

### BACKGROUND

Ultrasonic diagnostic devices are used for diagnosis of a tissue within a living body, for example, and are very useful especially in diagnosis of a fetus. However, because a fetus itself is small and the heart thereof is also very small, diagnosis of the heart of a fetus using ultrasonic diagnostic devices is extremely difficult. In M-mode measurement and Doppler measurement using an ultrasonic diagnostic device, for example, precise setting of a cursor with respect to the heart having a very small size is difficult. Under these circumstances, various techniques concerning diagnosis of a fetus using an ultrasonic diagnostic device have been proposed. Patent Document 1, for example, proposes an epoch-making technique for obtaining information concerning the heartbeat of a fetus, from which body shift information has been subtracted, based on motion information of the heart.

Indexes known in diagnosis of a fetus during the intermediate through later stages of pregnancy include Fractional Shortening (FS) of the left ventricle of the heart. An ultrasonic diagnostic device can measure FS of the heart using an M-mode function. However, while measurement lines in M-mode are normally fixed with respect to a probe, the fetal heart may move with respect to the probe, due to body motions of the fetus, respiration of the mother, and other influences, making highly precise FS measurement by M mode difficult to perform.

Attempts to measure FS while tracking the motion of the fetal heart through an image tracking technique using an ultrasonic image also reveal the difficulty in highly precise tracking of measurement locations necessary for the FS measurement, due to influences of motions of papillaris muscles and the mitral valve, for example.

### CITATION LIST

### Patent Literature

### [Patent Document 1] JP 2013-198636 A

### SUMMARY

### Technical Problem

The present invention was made in consideration of the background art described above and is directed to increasing the accuracy in tracking a motion of a measurement point concerning the heart of a fetus (fetal heart).

### Solution to Problem

In accordance with a preferable aspect, an ultrasonic diagnostic device includes a probe configured to transmit and receive an ultrasonic wave, a transmitter and receiver unit configured to control the probe to obtain a received signal of an ultrasonic wave from an interior of a living body, an image forming unit configured to form an ultrasound image including a heart of a fetus based on the received signal of an ultrasonic wave, and a tracking processing unit configured to track a motion of a measurement point concerning the heart of fetus within the ultrasound image. The tracking processing unit is configured to execute tracking processing in a plurality of modes within a tracking period, to thereby track the motion of the measurement point within the tracking period based on a plurality of tracking results obtained by the tracking processing in the plurality of modes.

In the above device, the tracking processing unit executes tracking processing in a plurality of modes within a tracking period. The tracking processing in a plurality of modes preferably has different processing modes. For example, the plurality of modes of the tracking processing preferably include different time directions and different correlation operations.

The above device tracks a motion of the measurement point based on a plurality of tracking results obtained by the tracking processing in the plurality of modes. This structure increases the accuracy in tracking concerning the motion of the measurement point by, for example, placing a higher priority to a preferred mode among the plurality of modes of the tracking processing in accordance with the tracking status.

In a specific preferable example, the tracking processing unit is configured to execute tracking processing in a forward direction and tracking processing in a backward direction with respect to time progress within the tracking period and to integrate a result of tracking in the forward direction and a result of tracking in the backward direction, thereby tracking the motion of the measurement point within the tracking period.

In a specific preferable example, after setting the tracking period to a period from a characteristic time phase to another characteristic time phase appearing periodically in an expansion and contraction motion of the heart of a fetus, the tracking processing unit is configured to execute tracking processing in the forward direction from one characteristic time phase, which is an origin, toward another characteristic time phase, and tracking processing in the backward direction from the another time phase, which is an origin, toward the one characteristic time phase, within the tracking period. The tracking processing unit is configured to further apply weighted summation to the tracking result in the forward direction and the tracking result in the backward direction according to a ratio corresponding to a distance with respect to time from the characteristic time phase which is an origin, thereby tracking the motion of the measurement point within the tracking period.

In a specific preferable example, the tracking processing unit is configured to execute tracking processing based on autocorrelation and tracking processing based on cross-correlation within the tracking period, and to integrate a tracking result based on autocorrelation and a tracking result based on cross-correlation, thereby tracking the motion of the measurement point within the tracking period.

In a specific preferable example, after setting a period from a characteristic time phase to another characteristic time phase appearing periodically in an expansion and contraction motion of the heart of a fetus to the tracking period, the tracking processing unit is configured to apply weighted summation to a tracking result based on the autocorrelation and a tracking result based on the cross-correlation with a ratio of the autocorrelation being increased as the characteristic time phase approaches with respect to time, thereby tracking the motion of the measurement point within the tracking period.

In a specific preferable example, the tracking processing unit is configured to execute tracking processing for each of a plurality of tracking points including a measurement point and an auxiliary point of the measurement point within the tracking period, and to track a motion of the measurement point within the tracking period based on a plurality of tracking results obtained from the plurality of tracking points.

In a specific preferable example, the tracking processing unit is configured to track the motion of the measurement point based on dynamic programming using the plurality of tracking results obtained from the plurality of tracking points.

In a specific preferable example, the ultrasonic diagnostic device is configured to track a motion of each of two measurement points set in the heart of a fetus in an ultrasound image and to derive a variation with respect to time concerning a distance between the two measurement points over at least one period of a heartbeat of a fetus.

In a specific preferable example, the ultrasonic diagnostic device is configured to calculate an FS value regarding the heart of the fetus based on the variation with respect to time concerning the distance between the two measurement points set in the heart of the fetus.

### Advantageous Effects of Invention

The present invention increases the accuracy in tracking the motion of a measurement point concerning the fetal heart.

### BRIEF DESCRIPTION OF DRAWINGS

FIG. 1 is a diagram illustrating a whole structure of an ultrasonic diagnostic device according to a preferred embodiment of the present invention.

FIG. 2 is a chart illustrating a specific example waveform of heartbeat obtained in a heartbeat measuring unit.

FIG. 3 is a diagram illustrating specific example tracking points set by a tracking point setting unit.

FIG. 4 explains specific example tracking processing concerning each tracking point.

FIG. 5 explains specific example tracking of measurement points using a plurality of auxiliary points.

FIG. 6 illustrates a specific example for combining tracking results in forward and backward directions.

FIG. 7 illustrates a specific example for combining tracking results based on autocorrelation and cross-correlation.

FIG. 8 is a chart for explaining example calculation of fractional shortening (FS) of the left ventricle.

### DESCRIPTION OF EMBODIMENTS

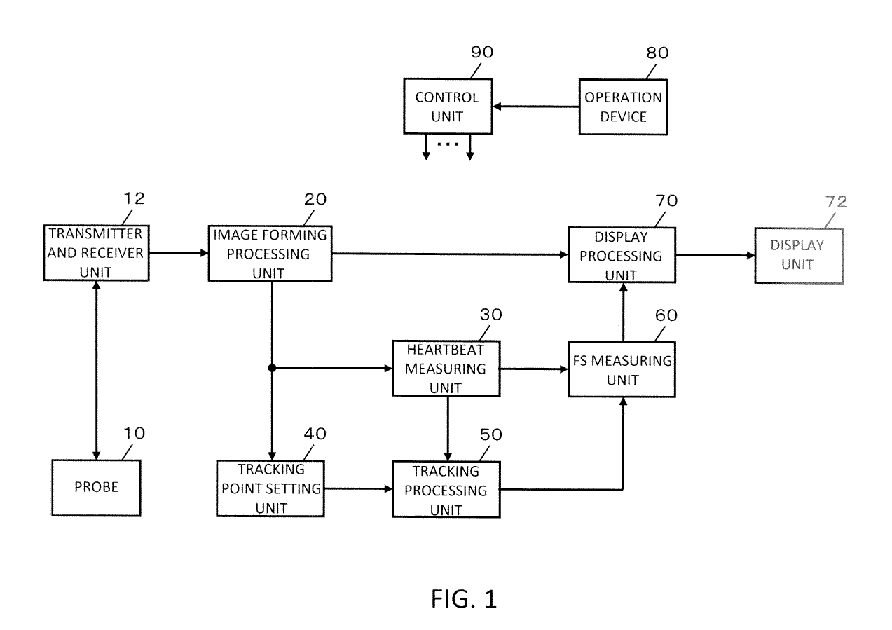

FIG. 1 is a diagram illustrating a whole structure of an ultrasonic diagnostic device according to a preferred embodiment of the present invention. A probe 10 is an ultrasound probe which transmits and receives ultrasonic waves to and from a diagnostic region including a fetus. The probe 10 includes a plurality of transducer elements for transmitting and receiving ultrasonic waves. The plurality of transducer elements are transmission-controlled by a transmitter and receiver unit 12 to form a transmitting beam. The plurality of transducer elements further receive ultrasonic waves from the diagnostic region, and output a signal obtained from the received ultrasonic waves to the transmitter and receiver unit 12. The transmitter and receiver unit 12 then forms a received beam to obtain a received signal (echo data). To transmit and receive the ultrasonic waves, a technique, such as transmission aperture synthesis, may be used.

An image forming unit 20, based on the received signal obtained from the transmitter and receiver unit 12, forms image data of an ultrasound image. The image forming unit 20 applies, to the received signal, signal processing including gain correction, logarithmic compression, wave detection, contour enhancement, filter processing, and other processing, as necessary, to form, for example, image data of a tomographic image (B-mode image) showing a fetus, for each of a plurality of frames (for each time phase).

The image data of a tomographic image formed in the image forming unit 20 are temporarily stored in a cine memory, for example. The cine memory may be included in the image forming unit 20 or may be disposed externally to the image forming unit 20. The image data formed in the image forming unit 20, such as the image data stored in the cine memory, undergo display processing by a display processing unit 70, and a tomographic image corresponding to the image data is displayed on a display unit 72.

A heartbeat measuring unit 30, a tracking point setting unit 40, and a tracking processing unit 50 process the image data formed in the image forming unit 20; that is, the image data stored in the cine memory, for example. The heartbeat measuring unit 30, based on the image data, measures heartbeats (expanding and contracting motion of the heart) of a fetus. The tracking point setting unit 40 sets a plurality of tracking points necessary for measurement concerning the fetal heart, in the image data of the tomographic image. The tracking processing unit 50 tracks the motion of each tracking point over a plurality of frames of the image data.

An FS measuring unit 60, based on the tracking results concerning the plurality of tracking points, calculates fractional shortening (FS) of the left ventricle serving as an index for diagnosis concerning the fetal heart. The calculated FS is displayed, via the display processing unit 70, on the display unit 72. Processing performed in the heartbeat measuring unit 30, the tracking point setting unit 40, the tracking processing unit 50, and the FS measuring unit 60 will be further described in detail below.

A control unit 90 controls the whole ultrasonic diagnostic device illustrated in FIG. 1. An instruction received by a user via an operation device 80 is also reflected in the whole control performed by the control unit 90.

Among the elements (the units designated by reference numerals) illustrated in FIG. 1, each of the transmitter and receiver unit 12, the image forming unit 20, the heartbeat measuring unit 30, the tracking point setting unit 40, the tracking processing unit 50, the FS measuring unit 60, and the display processing unit 70 may be implemented by using hardware such as an electrical or electronic circuit or a processor, for example, and a device such as a memory may be used as required for the implementation. At least a part of the functions corresponding to the respective units described above may be implemented by a computer; that is, by cooperation of hardware such as a CPU, a processor, and a memory, and software (program) which regulates the operation of the CPU or the processor.

A preferable specific example of the display unit 72 is a liquid crystal display, for example. The operation device 80 can be implemented by at least one of a mouse, a keyboard, a trackball, a touch panel, and other switches. The control unit 90 can be implemented by cooperation of hardware such as a CPU, a processor, and a memory, and software (program) which regulates the operation of the CPU or the processor.

The whole structure of the ultrasonic diagnostic device illustrated in FIG. 1 has been described as above. Specific example processing in the ultrasonic diagnostic device will now be described in detail. The following description concerning the elements (the units denoted by reference numerals) illustrated in FIG. 1 uses the reference numerals in FIG. 1.

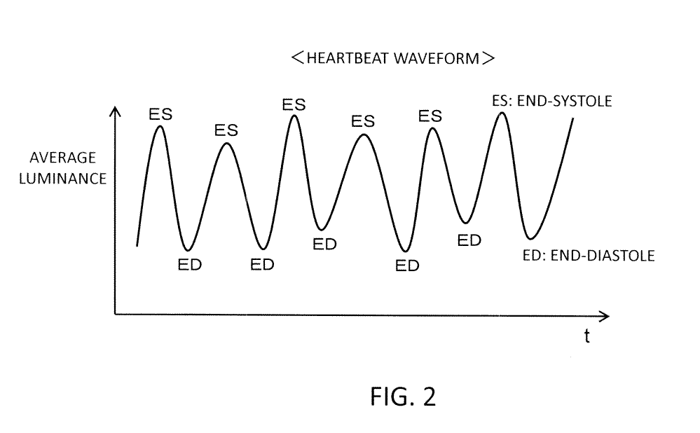

FIG. 2 is a chart illustrating a specific example heartbeat waveform obtained in the heartbeat measuring unit 30. FIG. 2 illustrates a heartbeat waveform indicating the average luminance which is an amplitude on the vertical axis, with the horizontal axis being a time axis (the frame number of image data over a plurality of frames).

To obtain the heartbeat waveform illustrated in FIG. 2, a region of interest is first set with respect to the fetal heart within the image data of a tomographic image. The region of interest is set in accordance with a user operation input via the operation device 80, for example. The user may operate the operation device 80 to set a location and a size of the region of interest such that the region of interest includes the fetal heart (particularly the cardiac wall), for example, while observing the tomographic image displayed on the display unit 72. Alternatively, the ultrasonic diagnostic device of FIG. 1 may analyze the image state within the tomographic image to set the region of interest in the fetal heart.

The region of interest is preferably set at a location where the motion of the fetal heart is easily detected. Specifically, the user designates the location and size of the region of interest such that the region of interest includes a portion of the fetal heart having a relatively high luminance, more particularly, the cardiac wall. Alternatively, the ultrasonic diagnostic device of FIG. 1 may determine a portion of the fetal heart having a relatively high luminance by image analysis processing such as binarization processing, for example, thereby determining the location and size of the region of interest. The region of interest may be set to other portions where the motion of the fetal heart can be easily detected.

After the region of interest is set, the heartbeat measuring unit 30, based on the image data within the region of interest, generates a fetal heartbeat waveform. The heartbeat measuring unit 30 calculates the average luminance (average of the luminance values) within the region of interest based on the image data within the region of interest, for example, and then calculates the average luminance over a plurality of frames obtained from the cine memory; that is, over a plurality of time points, thereby generating the heartbeat waveform illustrated in FIG. 2.

Due to a periodical expansion and contraction motion of the fetal heart, the average luminance varies with the expansion and contraction motion, and the heartbeat waveform in the specific example illustrated in FIG. 2, for example, is obtained. When the region of interest is set to include the cardiac wall, the ratio of the cardiac muscle within the region of interest increases with the contraction of the heart, resulting in an increased average luminance; whereas the ratio of the heart cavity within the region of interest increases with the expansion of the heart, resulting in a decreased average luminance. Thus, as in the specific example illustrated in FIG. 2, the luminance is periodically varied, so that a heartbeat waveform including periodical repetition of end-diastole ED and end-systole ES is obtained.

In place of the average luminance, a correlation value between time phases (between frames) of the image data may be used to generate the heartbeat waveform. Alternatively, after dividing the region of interest into a plurality of blocks and forming a heartbeat waveform for each block, a heartbeat waveform with high reliability may be selected from among a plurality of heartbeat waveforms corresponding to the plurality of blocks.

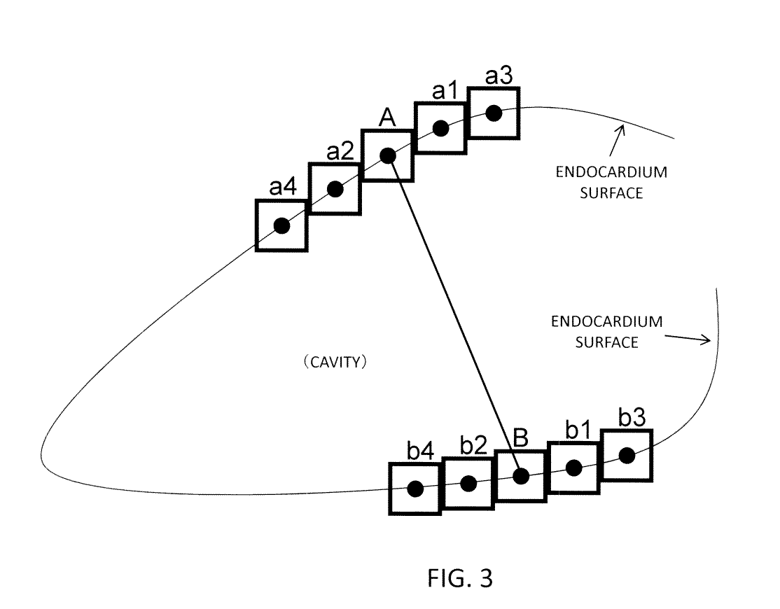

FIG. 3 is a diagram illustrating a specific example of a plurality of tracking points set by the tracking point setting unit 40. FIG. 3 illustrates the fetal heart (e.g., a cavity portion of the left ventricle) within a tomographic image. The ultrasonic diagnostic device illustrated in FIG. 1 has various measuring functions concerning the fetal heart, and particularly has a function preferred in measurement of the fractional shortening (FS) of the left ventricle. For FS measurement, two measurement points A and B are set with respect to the heart (e.g., the left ventricle).

The two measurement points A and B are set in accordance with a user operation input through the operation device 80, for example. A user designates locations of the two points at which FS measurement is to be performed, while observing the tomographic image on the display unit 72, for example. The tracking point setting unit 40 sets the measurement point A at one of the two locations designated by the user and sets the measurement point B at the other location.

It is desirable that the measurement point A and the measurement point B are set on an endocardium surface of the heart (an interface between the cardiac wall and the cavity). For example, the ultrasonic diagnostic device in FIG. 1 may detect the endocardium surface of the heart within a tomographic image using image analysis processing such as binarization processing, for example, and, without designation by a user or with the location designated by the user being modified, may determine the measurement point A and the measurement point B. The measurement point A and the measurement point B may be set at other locations where the motion of the fetal heart is easily detected.

Upon setting the two measurement points A and B, the tracking point setting unit 40 sets a plurality of auxiliary points around each of the measurement points A and B. As illustrated in FIG. 3, for example, for each of a plurality of frames, the tracking point setting unit 40 sets a plurality of auxiliary points (a1 to a4) in the vicinity of the measurement point A, and sets a plurality of auxiliary points (b1 to b4) in the vicinity of the measurement point B.

The tracking point setting unit 40 sets the plurality of auxiliary points (a1 to a4) on a straight line passing through the measurement point A and normal to a straight line AB extending through the measurement point A and the measurement point B. The distance between the measurement point A and each auxiliary point and the interval of a plurality of points which consists of the measurement point A and the plurality auxiliary points (a1 to a4) may be set to a predetermined value or adjusted as appropriate. The plurality of auxiliary points (a1 to a4) may be set on a straight line passing through the measurement point A and substantially parallel to the endocardium surface (circumscribing a boundary corresponding to the endocardium surface, for example).

The tracking point setting unit 40 sets the plurality of auxiliary points (b1 to b4) on a straight line passing through the measurement point B and normal to the straight line AB extending through the measurement point A and the measurement point B. The distance between the measurement point B and each auxiliary point and the interval of a plurality of points which consist of the measurement point B and the plurality auxiliary points (b1 to b4) may be set to a predetermined value or adjusted as appropriate. The plurality of auxiliary points (b1 to b4) may be set on a straight line passing through the measurement point B and substantially parallel to the endocardium surface (circumscribing a boundary corresponding to the endocardium surface, for example).

Once the measurement point A and the associated auxiliary points (a1 to a4), and the measurement point B and the associated auxiliary points (b1 to b4) are set, with all of these points (ten points in the specific example of FIG. 3) being designated as tracking points, the tracking processing unit 50 tracks a motion of each tracking point.

For tracking processing concerning each tracking point, a template is set for each tracking point. In FIG. 3, for example, a rectangle drawn by a solid line enclosing each tracking point indicated by a solid circle is a specific example template. The size of the template is set, for example, to M×N (M and N are natural numbers); M pixels in the horizontal direction and N pixels in the vertical direction.

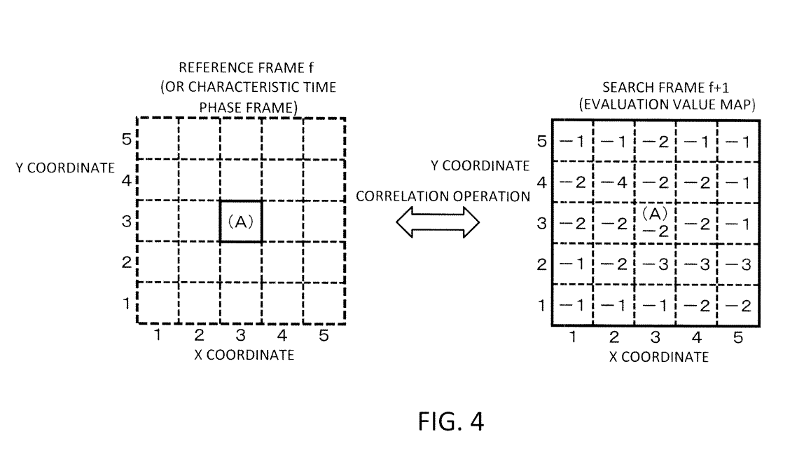

FIG. 4 is a diagram for explaining specific example tracking processing concerning each tracking point. FIG. 4 illustrates specific example tracking processing concerning the measurement point A as a representative example among a plurality of tracking points, and a destination of movement concerning the measurement point A in a reference frame f is located within a search frame f+1. In the specific example in FIG. 4, the reference frame f and the search frame f+1 are adjacent to each other with respect to time (with respect to the time phase). In place of the reference frame f, a frame at a characteristic time phase, such as end-diastole or end-systole, may be used as a reference.

In the specific example illustrated in FIG. 4, the measurement point A is at a location corresponding to coordinates (3, 3); that is, a location where the X coordinate is 3 and the Y coordinate is 3, in the reference frame f. For example, the measurement point A is at the center of a rectangle region corresponding to the coordinates (3, 3) enclosed by a solid line in FIG. 4. The rectangle region corresponds to the template of the measurement point A.

Further, in the search frame f+1, a search region is set to include a region (A) corresponding to the measurement point A, for example. In the specific example illustrated in FIG. 4, the search region is a region, in the search frame f+1, having X coordinates 1 to 5 and Y coordinates 1 to 5. Various known methods can be used for setting the search region. A whole image in the search frame f+1 may be used as the search region.

The tracking processing unit 50 moves the template of the measurement point A in the search region in the search frame f+1, and meanwhile performs a correlation operation to calculate a correlation evaluation value, based on a plurality of pixels within the template of the reference frame f at each moving location and a plurality of pixels at each moving location in the search frame f+1. In the specific example illustrated in FIG. 4, the evaluation value is calculated for each coordinate within the search frame f+1, and an evaluation value map indicating a distribution of evaluation values within the search region in the search frame f+1 is formed.

The correlation evaluation value is a numerical value indicating the degree of correlation (degree of similarity) between image data items. In the specific example illustrated in FIG. 4, the evaluation values are negative (−) numerical values, and the smaller the evaluation value (i.e., the greater the absolute value), the greater the degree of similarity. The evaluation values shown in FIG. 4 are only one specific example, and various known mathematical formulas related to the correlation operation can be used to calculate the evaluation values.

The tracking processing unit 50 executes the tracking processing described above with reference to FIG. 4 for each of the plurality of tracking points to obtain, for each tracking point, an evaluation value map in the search frame f+1. More specifically, similar to the evaluation value map concerning the measurement point A (FIG. 4), an evaluation value map is further calculated for each of the auxiliary points (a1 to a4). In addition, an evaluation value map concerning the measurement point B and an evaluation value map concerning each of the auxiliary points (b1 to b4) are also calculated.

To track the motion of the measurement point A, the tracking processing unit 50 uses, in addition to the evaluation value map concerning the measurement point A, the evaluation value maps concerning the plurality of auxiliary points (a1 to a4). Further, to track the motion of measurement point B, the tracking processing unit 50 uses, in addition to the evaluation value map concerning the measurement point B, the evaluation value maps concerning the plurality of auxiliary points (b1 to b4).

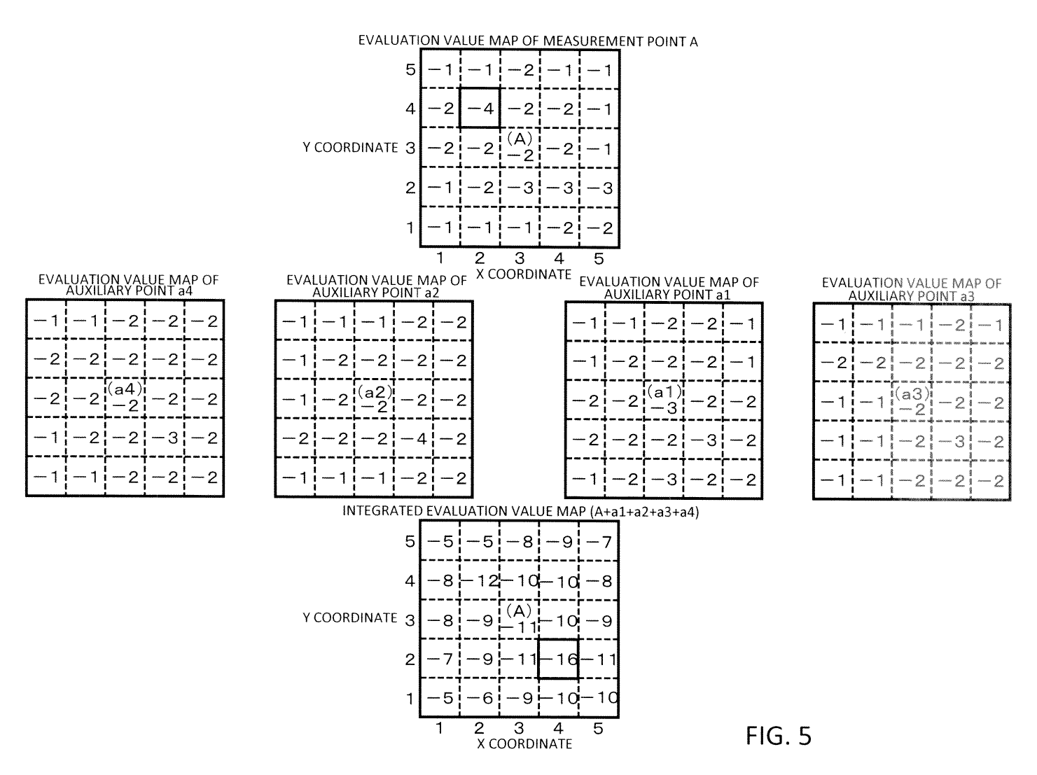

FIG. 5 explains specific example tracking of a measurement point using a plurality of auxiliary points. FIG. 5 illustrates a specific example tracking concerning the measurement point A, as a representative example of the two measurement points A and B.

To track the motion of the measurement point A, the tracking processing unit 50 uses, in addition to the evaluation value map concerning the measurement point A, the evaluation value maps concerning the auxiliary points (a1 to a4). FIG. 5 illustrates specific example evaluation value maps of the measurement point A and the auxiliary points (a1 to a4) in the same search frame (e.g., the search frame f+1 in FIG. 4).

The evaluation value map of each tracking point is an index for identifying a destination of movement (the location in the search frame) of the tracking point. In the specific example illustrated in FIG. 5, the evaluation value map of the measurement point A indicates that the evaluation value is the smallest at coordinates (2, 4); that is, at a location where the X coordinate is 2 and the Y coordinate is 4, and therefore that it is highly likely that the destination of movement of the measurement point A is at coordinates (2, 4). However, because this evaluation value map is obtained based on the correlation operation with respect to the measurement point A only, the smallest evaluation value may be obtained at an image location which is coincidentally identical (approximate) to the measurement point A, for example. Thus, a true destination of movement of the measurement point A may fail to be detected, leading to erroneous detection of the destination of movement.

To reduce, and more preferably eliminate, the possibility of such erroneous detection, the tracking processing unit 50 uses the evaluation value maps concerning the plurality of auxiliary points (a1 to a4) concerning the measurement point A. Because the plurality of auxiliary points (a1 to a4), which are set near the measurement point A, are likely to move in manners similar to the measurement point A, the destinations of movements of the auxiliary points (a1 to a4) are considered when detecting the destination of movement of the measurement point A.

Specifically, summation processing is applied to the evaluation value map of the measurement point A and the evaluation value maps of the plurality of auxiliary points (a1 to a4) to calculate an integrated evaluation value map concerning the measurement point A. While the measurement point A and the plurality of auxiliary points (a1 to a4) are disposed at different locations within a real space (see FIG. 3), for the purpose of summation of the evaluation value maps, the measurement point A and the plurality of auxiliary points (a1 to a4) are superposed with each other at the same location.

For example, the evaluation maps of the auxiliary points (a1 to a4) are translated such that the center of each evaluation value map concerning each of the auxiliary points (a1 to a4) (i.e., the location of each auxiliary point before moving) is superposed on the center of the evaluation map of the measurement point A (the location of the measurement point A before moving); that is, the location at coordinates (3, 3) in the specific example in FIG. 5, thereby superposing these evaluation value maps (five evaluation value maps in the specific example of FIG. 5). For each coordinate, the evaluation value of the measurement point A and the evaluation values of the plurality of auxiliary points (a1 to a4) are summed, so that an integrated evaluation value map indicating the evaluation value after summation for each coordinate can be obtained.

The evaluation value map concerning only the measurement point A illustrated in FIG. 5 indicates the smallest evaluation value at coordinates (2, 4); whereas the integrated evaluation map reflecting the motions of the plurality of auxiliary points (a1 to a4) indicates the destination of movement of the measurement point A at coordinates (4, 2). More specifically, in the specific example of FIG. 5, while, with the evaluation value map concerning only the measurement point A, the destination of movement of the measurement point A; that is, the coordinates (2, 4), may be erroneously detected, in the integrated evaluation value map, the coordinates (4, 2), which should be the proper destination of movement of the measurement point A, can be detected by reflecting the motions of the auxiliary points (a1 to a4) that are highly likely to be similar to the motion of the measurement point A.

Dynamic programming may be used for summation of the evaluation value map concerning the measurement point A and the evaluation value maps of the auxiliary points (a1 to a4). Processing which will be described below is performed when dynamic programming is used.

In dynamic programming, the evaluation value maps are summed in the following order: the auxiliary point a4, the auxiliary point a2, the measurement point A, the auxiliary point a1, and the auxiliary point a3 (the order of arrangement illustrated in FIG. 3), for example.

In summation of the evaluation value map of the auxiliary point a4 and the evaluation value map of the auxiliary point a2, with respect to a noted point at the coordinates (4, 2) in the evaluation value map of a2; that is, a location where X coordinate is 4 and Y coordinate is 2, for example, the following operation is executed according to the processing without using dynamic programming described above: simply summing the evaluation value for the coordinates (4, 2) in the evaluation value map of the auxiliary point a2 and the evaluation value for the same coordinates (4, 2) in the evaluation value map of the auxiliary point a4.

In the processing using dynamic programming, on the other hand, with respect to a noted point at coordinates (4, 2) in the evaluation value map of the auxiliary point a2, the following operation is executed: setting a search region to a neighboring range including this noted point; that is, a range of +1 to −1 in the X and Y directions about the center at the coordinates (4, 2); more specifically, to a range where coordinates along the X axis are 3 to 5 and coordinates along the Y axis are 1 to 3; searching this search range in the evaluation map of a4 for the best evaluation value (the smallest evaluation value); and summing this best evaluation value (the smallest evaluation value) in the search range of the auxiliary point a4 to the evaluation value at the noted point of the auxiliary point a2.

Similar to the specific example summation processing with respect to the noted point at the coordinates (4, 2) for the auxiliary point a2, the summation processing described above is executed for each of the noted points at all the coordinates within the evaluation value map of the auxiliary point a2. The evaluation value map of the auxiliary point a4 and the evaluation value map of the auxiliary point a2 are thus summed, and the evaluation values in the evaluation value map of the auxiliary point a2 are updated to the evaluation values after summation.

Then, the updated evaluation value map of the auxiliary point a2 and the evaluation value map of the measurement point A are summed. The specific example processing described above is also used for this summation. Specifically, a noted point is set to each of all the coordinates within the evaluation value map of the measurement point A, the search range including the noted point concerning each coordinate is searched in the evaluation value map of the auxiliary point a2, and the best valuation value (the smallest updated evaluation value) within the search range is added to the evaluation value of the measurement point A. The updated evaluation value map of the auxiliary point a2 and the evaluation value map of the measurement point A are thus summed, and the evaluation values in the evaluation value map of the measurement point A are updated to the evaluation values after summation.

The updated evaluation value map of the measurement point A and the evaluation value map of the auxiliary point a1 are further summed according to the specific example described above, and the evaluation value map of the auxiliary point a1 is updated. Further, the updated evaluation value map of the auxiliary point a1 and the evaluation value map of the auxiliary point a3 are further summed according to the specific example described above, and the evaluation value map of the auxiliary point a3 is updated.

The updated evaluation value map of the auxiliary point a3 thus obtained corresponds to an evaluation value map which has traced the optimal evaluation values in the evaluation maps of the auxiliary point a4, the auxiliary point a2, the measurement point A, the auxiliary point a1, and the auxiliary point a3. The coordinates having the best evaluation value (the smallest evaluation value after update) in the updated evaluation value map of the auxiliary point a3 are then designated as a selected point of the auxiliary point a3.

Subsequent to the determination of the selected point of the auxiliary point a3, a selected point of the auxiliary point a1 is determined. According to the summation processing described above, to obtain the updated evaluation value at the selected point of the auxiliary point a3, the search range including that selected point (noted point) in the evaluation value map of the auxiliary point a1 has been searched. Therefore, the search range including the selected point of the auxiliary point a3 is searched in the evaluation value map of the auxiliary point a1, and the best evaluation value (the smallest updated evaluation value) in the search range is designated as the selected point of the auxiliary point a1.

Once the selected point of the auxiliary point a1 is determined, selected points are sequentially determined in the following order: the measurement point A, the auxiliary point a2, and the auxiliary point a4. Specifically, the search range including the selected point of the auxiliary point a1 is searched in the evaluation value map of the measurement point A, and the best evaluation value (the smallest updated evaluation value) in the search range is designated as the selected point of the measurement point A. Further, the search range including the selected point of the measurement point A is searched in the evaluation value map of the auxiliary point a2, and the best evaluation value (the smallest updated evaluation value) in the search range is designated as the selected point of the auxiliary point a2. In addition, the search range including selected point of the auxiliary point a2 is searched in the evaluation value map of the auxiliary point a4, and the best evaluation value (the smallest updated evaluation value) in the search range is designated as the selected point of the auxiliary point a4.

In the specific example described above, the evaluation value maps are summed in the above-described order; that is, the auxiliary point a4, the auxiliary point a2, the measurement point A, the auxiliary point a1, and the auxiliary point a3, whereas the selected points are determined in the order of the auxiliary point a3, the auxiliary point a1, the measurement point A, the auxiliary point a2, and the auxiliary point a4. In contrast, the evaluation value maps may be summed in the order of the auxiliary point a3, the auxiliary point a1, the measurement point A, the auxiliary point a2, and the auxiliary point a4 and the selected points may be determined in the order of the auxiliary point a4, the auxiliary point a2, the measurement point A, the auxiliary point a1, and the auxiliary point a3.

As described above, the selected point is determined concerning each of the measurement point A and the auxiliary points (a1 to a4) according to dynamic programming. Then, coordinates at the selected point of the measurement point A, for example, are designated as the destination of movement of the measurement point A (the coordinates after moving). The destination of movement of the measurement point A may be determined based on a plurality of selected points concerning the measurement point A and the auxiliary points (a1 to a4). For example, the average location (mean values of coordinates in X axis and Y axis) concerning the plurality of selected points of the measurement point A and the auxiliary points (a1 to a4) may be designated as the destination of movement of the measurement point A.

Further, whether or not the destination of movement of the measurement point A obtained by the original evaluation value map (the evaluation value map prior to update by summation processing) of the measurement point A is appropriate may be determined based on the selected point of the measurement point A obtained according to dynamic programming to determine a proper destination of movement of the measurement point A. With a neighboring range including the selected point of the measurement point A obtained according to dynamic programming; more specifically, a range of +1 to −1 about the center at the coordinates of the selected point in the X and Y directions, being designated as a search range, for example, the original evaluation map of the measurement point A is searched. If the destination of movement of the measurement point A obtained only from the original evaluation value map of the measurement point A (coordinates (2, 4) in the specific example of FIG. 5) is present within the search range, the destination of movement is determined as the proper destination of movement of the measurement point A; whereas if such a destination is not present within the search range, the selected point of the measurement point A obtained according to dynamic programming is determined as the proper destination of movement. If the destination of movement obtained only from the original evaluation value map of the measurement point A is not present within the search range, the proper destination of movement of the measurement point A may be determined based on that destination of movement and the selected point. For example, the mean location (midpoint) of the destination of movement obtained only from the original evaluation value map of the measurement point A and the selected point of the measurement point A based on dynamic programming may be determined as the proper destination of movement of the measurement point A.

The tracking processing unit 50, on detecting the location of the measurement point A within the search frame f+1 according to the processing described above with reference to FIGS. 4 and 5, for example, further executes the processing described above with reference to FIGS. 4 and 5 in a frame f+2 in the next time phase, which is now set as a search frame, and detects the location of the measurement point B in the frame f+2. In this manner, the tracking processing unit 50 tracks the motion of the measurement point A over a plurality of frames of the image data stored in the cine memory, for example, and further tracks the motion of the measurement point B over a plurality of frames in a manner similar to the tracking of the measurement point A.

To track the motions of the measurement point A and the measurement point B over a plurality of frames, the tracking processing unit 50 may track the motions of the measurement point A and the measurement point B within a period corresponding to one heartbeat, for example, based on a plurality of tracking results obtained by tracking processing in a plurality of modes within that period. For example, the tracking processing unit 50 may execute tracking processing in forward and backward directions with respect to the progress of time within a period corresponding to one heartbeat, to track the motions of the measurement point A and the measurement point B within the period based on integration of the tracking result in the forward direction and the tracking result in the backward direction.

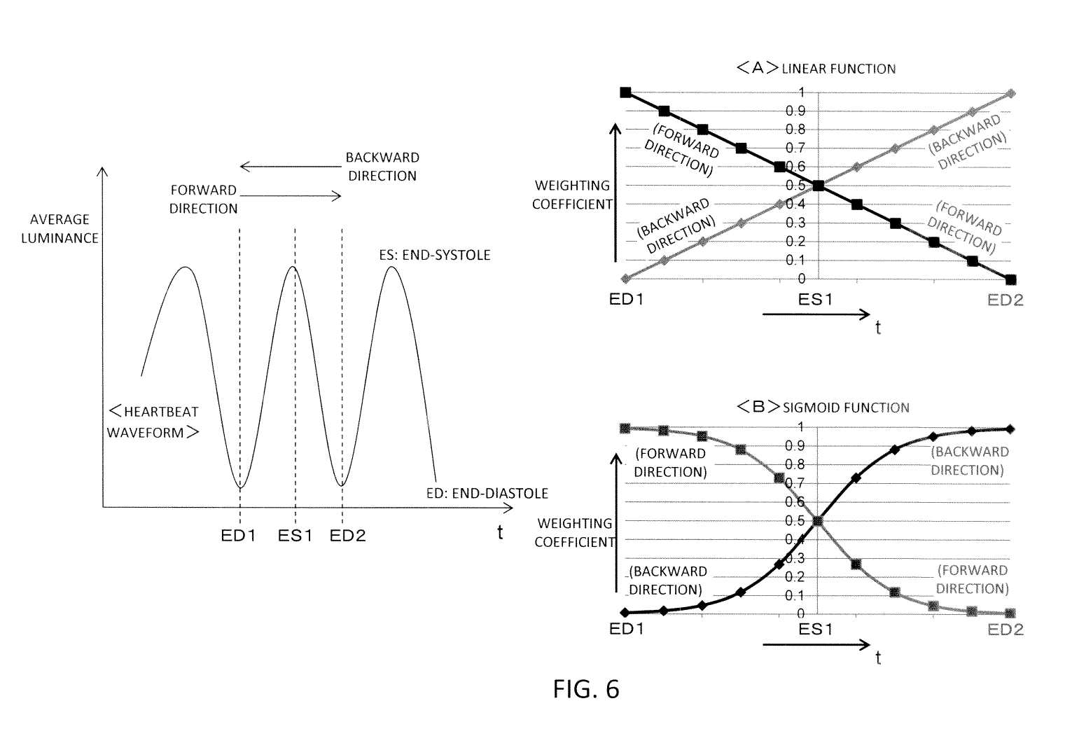

FIG. 6 is a diagram illustrating a specific example for combining tracking results in the forward and backward directions. FIG. 6 illustrates a specific example heartbeat waveform (see FIG. 2) formed in the heartbeat measuring unit 30. The heartbeat measuring unit 30 detects, within a heartbeat waveform in which the average luminance is varied periodically, a time phase of the end-systole ES with the maximum average luminance and a time phase of the end-diastole ED with the minimum average luminance.

The tracking processing unit 50 executes tracking processing in the forward direction and the backward direction within one heartbeat period specified by the time phase of the end-diastole ED or the time phase of the end-systole ES. FIG. 6 illustrates a specific example in which the processing is executed in one target heartbeat period from the end-diastole ED1, via the end-systole ES1, to the next end-diastole ED2.

The tracking processing unit 50 first derives, concerning a measurement point A within a frame of the end-diastole ED1, a location within a frame of the end-diastole ED2 which corresponds to the measurement point A, for example. The tracking processing unit 50, for example, specifies a location within the frame of the end-diastole ED2 corresponding to the measurement point A, according to pattern matching based on a correlation operation using a template corresponding to the measurement point A in the end-diastole ED1. The tracking processing unit 50 may specify the location of the measurement point A within the frame of the end-diastole ED2 based on the processing reflecting the motions of the auxiliary points (a1 to a4) concerning the measurement point A (see FIG. 5).

The tracking processing unit 50 then tracks the motion of the measurement point A at the end-diastole ED1 in the forward direction. More specifically, the tracking processing unit 50 tracks the motion of the measurement point A at the end-diastole ED1 for a plurality of time phases; that is, a plurality of frames, in the time direction from the end-diastole ED1, via the end-systole ES1, toward the end-diastole ED2.

The tracking processing unit 50 further tracks the motion of the measurement point A at the end-diastole ED2 in the backward direction. Specifically, the tracking processing unit 50 tracks the motion of the measurement point A at the end-diastole ED2 for a plurality of time phases; that is, a plurality of frames, in the time direction from the end-diastole ED2, via the end-systole ES1, toward the end-diastole ED1.

To track the motion of the measurement point A in the forward and backward directions, it is desirable that the tracking processing unit 50 should track the location of the measurement point A within each frame, according to the processing reflecting the motions of a plurality of auxiliary point (a1 to a4) concerning the measurement point A (see FIG. 5).

The tracking processing unit 50 then applies weighted summation to the tracking result in the forward direction and the tracking result in the backward direction at ratios in accordance with a distance with respect to time from the end-diastole ED1 (or the end-diastole ED2) which is the origin of tracking, thereby tracking the motion of the measurement point A within a period of one heartbeat from the end-diastole ED1 to the end-diastole ED2.

For example, for each time phase (each frame) of a plurality time phases (a plurality of frames), weighted summation is applied to a coordinate value of the measurement point A which is the tracking result in the forward direction and a coordinate value of the measurement point A which is the tracking result in the backward direction to calculate an integrated coordinate value of the measurement point A in that time phase (frame). Specifically, the integrated x coordinate is calculated according to the weighted summation concerning the x coordinate and the integrated y coordinate is calculated according to weighted summation concerning the y coordinate. FIG. 6 illustrates specific example weighting coefficients for the weighted summation.

The linear function shown in FIG. 6<A> shows a specific example in which the weighting coefficient is varied by a linear function in accordance with a distance with respect to time from the end-diastole ED1 (or the end-diastole ED2). More specifically, the weighting coefficient in the forward direction at the end-diastole ED1 is the maximum value, 1, which is linearly decreased as the distance from the end-diastole ED1 increases with respect to time and reaches the minimum value 0 at the end-diastole ED2. The weighting coefficient in the backward direction, on the other hand, is obtained by subtracting, from the maximum value 1, the weighting coefficient in the forward direction.

The linear function of FIG. 6<A> enables tracking of the measurement point A while reflecting the tracking result in the forward direction more as the distance to the end-diastole ED1 decreases and reflecting the tracking result in the backward direction more as the distance to the end-diastole ED2 decreases.

The sigmoid function shown in FIG. 6<B> shows a specific example in which the weighting coefficient is varied by a sigmoid function in accordance with a distance with respect to time from the end-diastole ED1 (or the end-diastole ED2). More specifically, the weighting coefficient in the forward direction at the end-diastole ED1 is the maximum value, 1, which is decreased in a curved manner according to the sigmoid function as the distance from the end-diastole ED1 increases with respect to time and reaches the minimum value, 0, at the end-diastole ED2. The weighting coefficient in the backward direction, on the other hand, is obtained by subtracting, from the maximum value 1, the weighting coefficient in the forward direction.

The sigmoid function of FIG. 6<B>, as compared to the linear function, reflects the tracking result in the forward direction further more in the vicinity of the end-diastole ED1 and reflects the tracking result in the backward direction further more in the vicinity of the end-diastole ED2. The curvature of the sigmoid function is preferably adjusted as appropriate.

The tracking processing unit 50 performs, with regard to the measurement point B, tracking processing in the forward direction and the backward direction similar to the processing performed with regard to the measurement point A described above, and applies weighted summation to the tracking result in the forward direction and the tracking result in the backward direction. The tracking processing unit 50 thus tracks the motion of the measurement point B during one heartbeat from the end-diastole ED1 to the end-diastole ED2.

The tracking processing unit 50 may further execute, during a period corresponding to one heartbeat, for example, tracking processing according to autocorrelation and tracking processing according to cross-correlation and integrate the tracking result based on the autocorrelation and the tracking result based on the cross-correlation, thereby tracking the motions of the measurement point A and the measurement point B during this period.

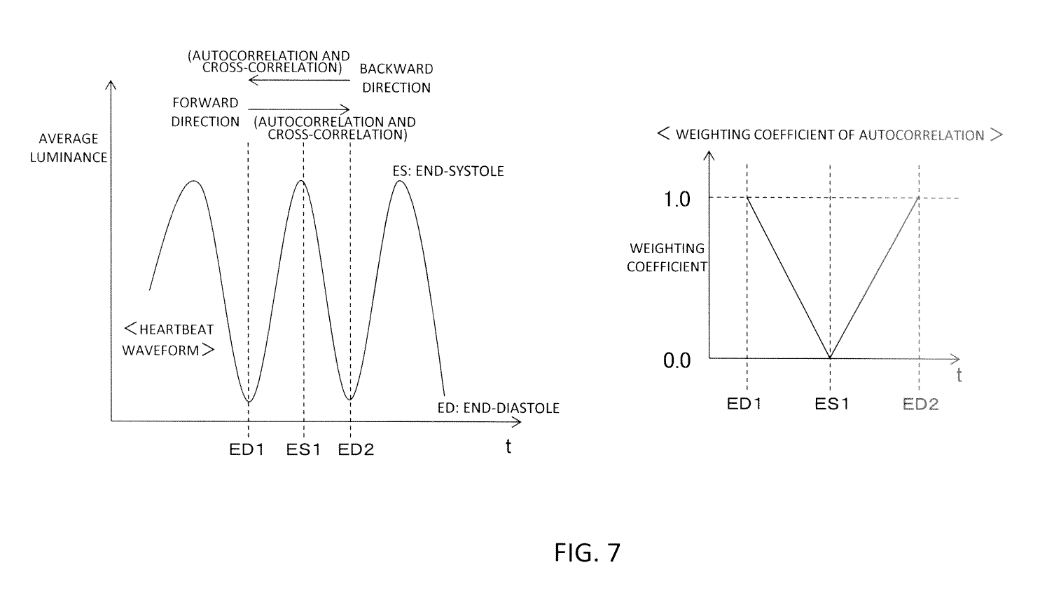

FIG. 7 is a diagram illustrating a specific example in which tracking results based on autocorrelation and cross-correlation are combined. FIG. 7 illustrates a specific example heartbeat waveform (see FIG. 2) formed by the heartbeat measuring unit 30.

The tracking processing unit 50 executes tracking processing according to autocorrelation and cross-correlation during one heartbeat specified by the time phase of the end-diastole ED or the time phase of the end-systole ES, for example. FIG. 7 illustrates a specific example for performing such tracking processing during one heartbeat period from the end-diastole ED1, via the end-systole ES1, to the next end-diastole ED2.

To track the motion of the measurement point A at the end-diastole ED1 in the forward direction (see FIG. 6), the tracking processing unit 50 executes both an autocorrelation operation and a cross-correlation operation. In the autocorrelation, with a frame at the end-diastole ED1 being used as a reference frame, the location corresponding to the measurement point A within each frame (search frame) is specified according to the correlation operation using a template of the measurement point A at the end-diastole ED1. In the cross-correlation, on the other hand, with respect to the frame f and the frame f+1 which are adjacent to each other, the location corresponding to the measurement point A in the frame f+1 (search frame) is specified according to the correlation operation using the template of the measurement point A in the frame f.

To track the motion of the measurement point A using the autocorrelation and the cross-correlation, the tracking processing unit 50 preferably tracks the location of the measurement point A in each frame, according to the processing based on dynamic programming with reference to the motions of the auxiliary points (a1 to a4) concerning the measurement point A (see FIG. 5). For example, in the tracking using the autocorrelation, evaluation values of the correlation (see FIG. 4) are calculated based on the operation of autocorrelation, and in the tracking using cross-correlation, evaluation values of the correlation are calculated based on the operation of cross-correlation.

The tracking processing unit 50 then executes weighted summation of the tracking result based on autocorrelation and the tracking result based on cross-correlation at ratios in accordance with a distance with respect to time from the end-diastole ED1 (or the end-diastole ED2), thereby tracking the motion of the measurement point A during one heartbeat period from the end-diastole ED1 to the end-diastole ED2.

The tracking processing unit 50, for example, executes weighted summation of the coordinate value of the measurement point A which is the tracking result based on autocorrelation and the coordinate value of the measurement point A which is the tracking result based on cross-correlation for each time phase (each frame) of a plurality of time phases (a plurality of frames) to calculate an integrated coordinate value of the measurement point A in the time phase (frame). Specifically, an integrated x coordinate is calculated according to the weighted summation concerning the x coordinate, and an integrated y coordinate is calculated according to the weighted summation concerning the y coordinate. FIG. 7 illustrates specific example weighting coefficients for the autocorrelation in the weighted summation.

In the specific example weighting coefficient for autocorrelation illustrated in FIG. 7, the weighting coefficient for autocorrelation is linearly varied in accordance with a distance with respect to time from the end-diastole ED1 (or the end-diastole ED2). More specifically, the weighting coefficient for autocorrelation at the end-diastole ED1 is the maximum value, 1, which is linearly decreased as the distance from the end-diastole ED1 increases with respect to time and reaches the minimum value, 0, at the end-systole ES1.

The weighting coefficient for autocorrelation then linearly increases from the end-systole ES1 toward the end-diastole ED2 and reaches the maximum value, 1, at the end-diastole ED2. The weighting coefficient for cross-correlation, on the other hand, is obtained by subtracting, from the maximum value 1, the weighting coefficient for autocorrelation.

The weighting coefficient for autocorrelation illustrated in FIG. 7 enables tracking of the measurement point A while reflecting the tracking result of autocorrelation more as the distance to the end-diastole ED1 or the end-diastole ED2 decreases and reflecting the tracking result of cross-correlation more as the distance from the end-diastole ED1 or the end-diastole ED2 increases.

To track the motion of the measurement point A based on autocorrelation and cross-correlation from the origin, the end-systole ES, the tracking result based on autocorrelation may be reflected more as the distance to the origin, which is the end-systole ES, decreases and the tracking result based on cross-correlation may be reflected more as the distance from the end-systole ES increases; that is, as the distance to the end-diastole ED decreases.

The tracking processing unit 50 also executes both the autocorrelation operation and the cross-correlation operation to track the motion of the measurement point A at the end-diastole ED2 in the backward direction (see FIG. 6). In autocorrelation, with a frame at the end-diastole ED2 being used as a reference frame, the location corresponding to the measurement point A within each frame (search frame) is specified according to the correlation operation using a template of the measurement point A at the end-diastole ED2. In the cross-correlation, on the other hand, with respect to the frame f and the frame f+1 which are adjacent to each other, the location corresponding to the measurement point A in the frame f (search frame) is specified according to the correlation operation using the template of the measurement point A in f+1.

The tracking processing unit 50 further executes, concerning the measurement point B, tracking processing based on autocorrelation and cross-correlation similar to that for the measurement point A described above and performs weighted summation with respect to the tracking result of the autocorrelation and the tracking result of the cross-correlation, thereby tracking the motion of the measurement point B in the forward direction and backward direction during one heartbeat period from the end-diastole ED1 to the end-diastole ED2.

After the tracking processing unit 50 tracks the motions of the measurement point A and the measurement point B at least during one heartbeat, and preferably for a plurality of heartbeats, the FS measuring unit 60 calculates the fractional shortening (FS) of the left ventricle, which is an index of diagnosis concerning the fetal heart.

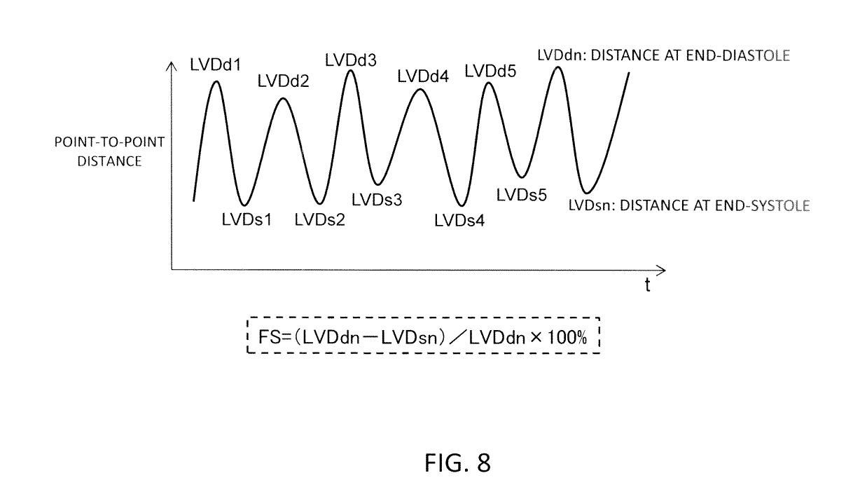

FIG. 8 is a chart for explaining example calculation of fractional shortening (FS) of the left ventricle. The FS measuring unit 60, based on the tracking results of the measurement point A and the measurement point B, calculates a distance between the measurement point A and the measurement point B (a point-to-point distance) for each of a plurality of time phases.

FIG. 8 illustrates a variation waveform of the point-to-point distance over the plurality of time phases. Because the fetal heart periodically expands and contracts, the measurement point A and the measurement point B (see FIG. 3) change with the expansion and contraction motion, and the variation waveform of the point-to-point distance as illustrated in the specific example in FIG. 8 is obtained. More specifically, as the point-to-point distance decreases with the contraction of the heart, the point-to-point distance is the minimum value LVDsn at end-systole; whereas as the point-to-point distance increases with the expansion of the heart, the point-to-point distance is the maximum value LVDdn at end-diastole. The point-to-point distance is varied periodically as in the specific example illustrated in FIG. 8, so that the variation waveform which repeats LVDsn and LVDdn periodically can be obtained.

The FS measuring unit 60 calculates the fractional shortening (FS) of the left ventricle according to the following formula, based on the point-to-point distance between the measurement point A and the measurement point B. The FS measuring unit 60 may calculate % FS (percent FS) by multiplying the result of Mathematical Formula 1 by 100 (percent).

FS=(LVDdn−LVDsn)/LVDdn Mathematical Formula 1

The FS(% FS) calculated in the FS measuring unit 60 is displayed on the display unit 72 via the display processing unit 70. The heartbeat waveform (FIG. 2) or the variation waveform of the point-to-point distance (FIG. 8) may be displayed on the display unit 72.

While embodiments of the present invention have been described, the embodiments described above are only illustrative in all respects, and do not limit the scope of the invention. The present invention includes various modifications without departing from its spirit.

### REFERENCE SIGNS LIST

10 probe, 12 transmitter and receiver unit, 20 image forming unit, 30 heartbeat measuring unit, 40 tracking point setting unit, 50 tracking processing unit, 60 FS measuring unit, 70 display processing unit, 72 display unit, 80 operation device, 90 control unit.

1. An ultrasonic diagnostic device, comprising:
a probe configured to transmit and receive an ultrasonic wave;
a transmitter and receiver unit configured to control the probe to obtain a received signal of an ultrasonic wave from an interior of a living body;
an image forming unit configured to form an ultrasound image including a heart of a fetus based on the received signal of an ultrasonic wave; and
a tracking processing unit configured to track a motion of a measurement point concerning the heart of fetus within the ultrasound image,
wherein
the tracking processing unit is configured to execute tracking processing in a plurality of modes within a tracking period, to track the motion of the measurement point within the tracking period based on a plurality of tracking results obtained by the tracking processing in the plurality of modes.

2. The ultrasonic diagnostic device according to claim 1, wherein
the tracking processing unit is configured to execute tracking processing in a forward direction and tracking processing in a backward direction with respect to time progress within the tracking period and to integrate a result of tracking in the forward direction and a result of tracking in the backward direction, thereby tracking the motion of the measurement point within the tracking period.

3. The ultrasonic diagnostic device according to claim 2, wherein,
the tracking processing unit is configured, after setting a period from a characteristic time phase to another characteristic time phase appearing periodically in an expansion and contraction motion of the heart of a fetus to the tracking period, to execute, within the tracking period, tracking processing in the forward direction from one characteristic time phase, which is an origin, toward another characteristic time phase and tracking processing in the backward direction from the another time phase, which is an origin, toward the one characteristic time phase, and to apply weighted summation to the tracking result in the forward direction and the tracking result in the backward direction according to a ratio corresponding to a distance with respect to time from the characteristic time phase which is an origin, thereby tracking the motion of the measurement point within the tracking period.

4. The ultrasonic diagnostic device according to claim 1, wherein
the tracking processing unit is configured to execute tracking processing based on autocorrelation and tracking processing based on cross-correlation within the tracking period, and to integrate a tracking result based on autocorrelation and a tracking result based on cross-correlation, thereby tracking the motion of the measurement point within the tracking period.

5. The ultrasonic diagnostic device according to claim 4, wherein
the tracking processing unit is configured to apply, after setting a period from a characteristic time phase to another characteristic time phase appearing periodically in an expansion and contraction motion of the heart of a fetus to the tracking period, weighted summation to a tracking result based on the autocorrelation and a tracking result based on the cross-correlation, with a ratio of the autocorrelation being increased as the characteristic time phase approaches with respect to time, thereby tracking the motion of the measurement point within the tracking period.

6. The ultrasonic diagnostic device according to claim 2, wherein
the tracking processing unit is configured to execute tracking processing based on autocorrelation and tracking processing based on cross-correlation within the tracking period, and to integrate a tracking result based on autocorrelation and a tracking result based on cross-correlation, thereby tracking the motion of the measurement point within the tracking period.

7. The ultrasonic diagnostic device according to claim 6, wherein
the tracking processing unit is configured to apply, after setting a period from a characteristic time phase to a characteristic time phase appearing periodically in an expansion and contraction motion of the heart of a fetus to the tracking period, weighted summation to a tracking result based on the autocorrelation and a tracking result based on the cross-correlation with a ratio of the autocorrelation being increased as the characteristic time phase is approaching with respect to time, thereby tracking the motion of the measurement point within the tracking period.

8. The ultrasonic diagnostic device according to claim 3, wherein
the tracking processing unit is configured to execute tracking processing based on autocorrelation and tracking processing based on cross-correlation within the tracking period, and to integrate a tracking result based on autocorrelation and a tracking result based on cross-correlation, thereby tracking the motion of the measurement point within the tracking period.

9. The ultrasonic diagnostic device according to claim 8, wherein
the tracking processing unit is configured to apply, after setting a period from a characteristic time phase to a characteristic time phase appearing periodically in an expansion and contraction motion of the heart of a fetus to the tracking period, weighted summation to a tracking result based on the autocorrelation and a tracking result based on the cross-correlation with a ratio of the autocorrelation being increased as the characteristic time phase is approaching with respect to time, thereby tracking the motion of the measurement point within the tracking period.

10. The ultrasonic diagnostic device according to claim 1, wherein
the tracking processing unit is configured to execute tracking processing for each of a plurality of tracking points including a measurement point and an auxiliary point of the measurement point within the tracking period, and to track a motion of the measurement point within the tracking period based on a plurality of tracking results obtained from the plurality of tracking points.

11. The ultrasonic diagnostic device according to claim 10, wherein
the tracking processing unit is configured to track the motion of the measurement point based on dynamic programming using the plurality of tracking results obtained from the plurality of tracking points.

12. The ultrasonic diagnostic device according to claim 1, wherein
a motion of each of two measurement points set in the heart of a fetus in an ultrasound image is tracked, and a variation with respect to time concerning a distance between the two measurement points is derived over at least one period of a heartbeat of a fetus.

13. The ultrasonic diagnostic device according to claim 12, wherein
an FS value regarding the heart of the fetus is calculated based on the variation with respect to time concerning the distance between the two measurement points set in the heart of the fetus.

14. The ultrasonic diagnostic device according to claim 6, wherein
a motion of each of two measurement points set in the heart of a fetus in an ultrasound image is tracked, and a variation with respect to time concerning a distance between the two measurement points is derived over at least one period of a heartbeat of a fetus.

15. The ultrasonic diagnostic device according to claim 14, wherein
an FS value regarding the heart of the fetus is calculated based on the variation with respect to time concerning the distance between the two measurement points set in the heart of the fetus.
